(19) United States
(12) Patent Application Publication
(10) Pub. No.: US 2012/0048283 A1
(43) Pub. Date: Mar. 1, 2012

(54) Foot Protection & Alignment Device

(76) Inventor: Edward Scott Cenek, Tarpon Springs, FL (US)

(21) Appl. No.: 12/862,835

(22) Filed: Aug. 25, 2010

(51) Int. Cl. A61F 5/00

(52) U.S. Cl. 128/882

(57) ABSTRACT

The Foot Protection & Alignment Device is protective footwear that keeps the foot in the proper position during the healing process. The device incorporates a multi-positional arch support to assist in positional alignment. The device also has a non-removable HDPE insert that is rigid, keeping the foot from flexing. The top of the device is fully adjustable, as is the heel, to accommodate feet of all widths. There are ABS hard plastic guards over the toes and mid-foot that are removable.

The Foot Protection & Alignment Device is comprised as follows:

(All Components are Shown in FIG. 1)

The sole of the footwear is ¼-⅜ inch thick and is a dense foam (denser than a “flip-flop” but not as dense as an athletic shoe sole) to absorb the impact of walking rather than have it transferred to the leg and hip. The underside of the sole is treaded to eliminate slipping. The sole is attached to the “upper” part of the footwear. The “upper” is comprised of a Velcro® compatible fabric sewn to a piece of chipboard. Also sewn to the chipboard are two straps of nylon covered foam (approx. 3/16″ thick) that attach together via a piece of Velcro® over the foot (this strap keeps the foot in the device properly). The Velcro® compatible fabric has a foam core (approx. ⅛″) and a nylon tricot backing The exposed edges of the fabric are finished with a bias tape or edging material. The center of the heal is split to allow adjustment for various foot widths. A piece of Velcro® secures the two heal sections into the proper position. The two sides of the “upper” are secured together, over the foot, via two Velcro® straps. An insert of ¼″ thick HDPE (High Density Poly Ethylene) is attached to the chipboard (to provide rigidity). A two millimeter thick piece of foam is attached to the exposed surface of the HDPE insert (pattern of upper “side”, HDPE insert, foam covering, chipboard and sole are represented in FIG. 3) (FIG. 2 shows a side view of the sole material, the HDPE insert, the chipboard and the 2 mm thick foam). A soft foam (urethane type—covered with a flannel type material and a small square of hard plastic on the underside) arch support (FIG. 4) is attached to the foam via self-adhesive Velcro® (the self-adhesive Velcro® adheres to the hard plastic and allows variation of placement). The device uses two protective guards, but comes with three (the two toe guards are interchangeable—one has an open front to allow clearance for the “pins” that doctors sometimes use and one has a closed front) and one over the midfoot. The toe guard with the open front attaches to the HDPE insert via two tab and slots (the tabs are on the HDPE and the slots are cut in the ABS toe guard) and the toe guard with the closed front has a tab and slot on the front and both sides. The tab and slots on both toe guards also act as an alignment system. Each toe guard also has a piece of self-adhesive Velcro® (on the underside) near the foot guard. The foot guard has vinyl trim on the bottom two edges and on the edge by the ankle. It also has a piece of Velcro® (on the underside) on the edge near the toe guard—a piece of mating Velcro® holds the foot guard to the toe guard. The foot guard has a piece of foam (approx. 3/16″ thick with a nylon covering) attached to the underside to keep the foot from touching the guard. FIG. 1 shows an exploded view of all parts involved.

* Velcro® is a registered trademark.
** Generic hook and loop fasteners will be substituted for Velcro.

### SPECIFICATIONS

When a human foot is injured, or has undergone a procedure or operation, it is important to keep the foot in the proper position while healing. Most of the post-procedure, or post-op shoes, only offer a stiff sole. It is vital to keep the main arch supported to eliminate, or minimize, future problems. While a rigid support is ideal, it is uncomfortable and therefore unrealistic. All available products leave the toes exposed and totally unprotected. In addition, most products are for medium to wide feet, eliminating use by narrow feet.

The upper section of the Foot Protection & Alignment Device is made of a Velcro° compatible fabric—the hook side of hook & loop fasteners sticks to it—and has a sole of high density foam that is textured on one side to prevent slipping. The sole is semi-shock absorbent therefore able to absorb the impact of walking. There is a non-removable insert of High Density Poly Ethylene that is non-flexible that keeps the foot from flexing. The device has an adjustable width heel, by way of a Velcro° type fastener which allows the device to be used on feet of various widths. There is an arch support in the device that is multi-positional and keeps the foot supported in the proper position during the healing process. The arch support is held in place by a hook and loop fastener. It has a piece of flat plastic molded into the bottom to give the self adhesive hook & loop a place to mount. The device has two removable protectors—one over the toes and one over the mid foot. The protector over the toes is held in place by three tab & slots while the mid foot protector rests in place and is secured by the upper parts of the device.

### SUMMARY OF FIGURES (DRAWINGS)

FIG. 1: exploded view of all components

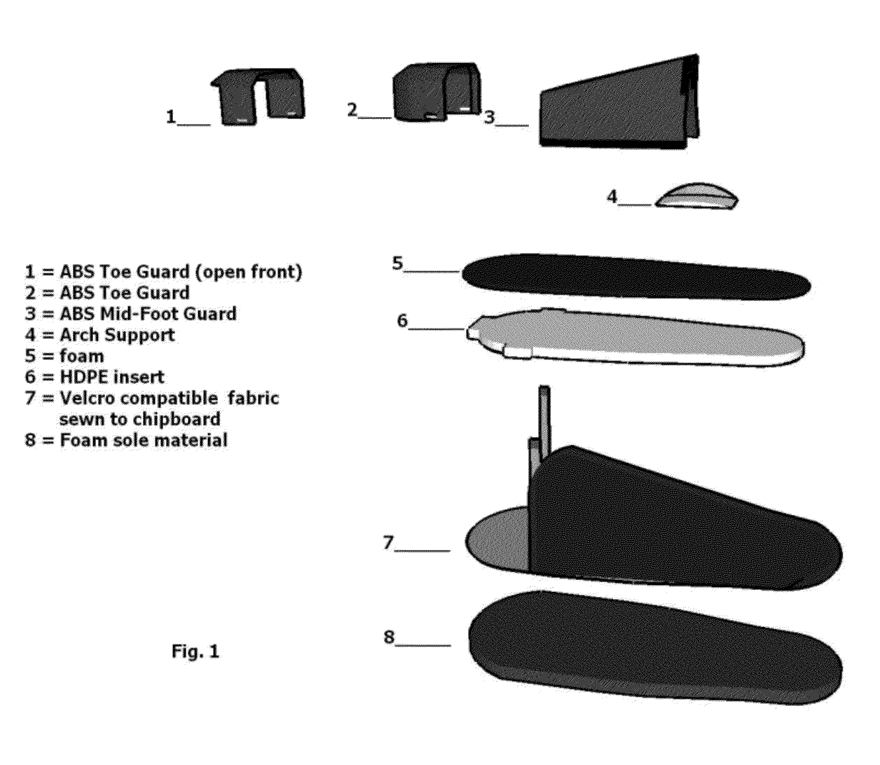

FIG. 2: side view of sole material, HDPE insert & foam covering

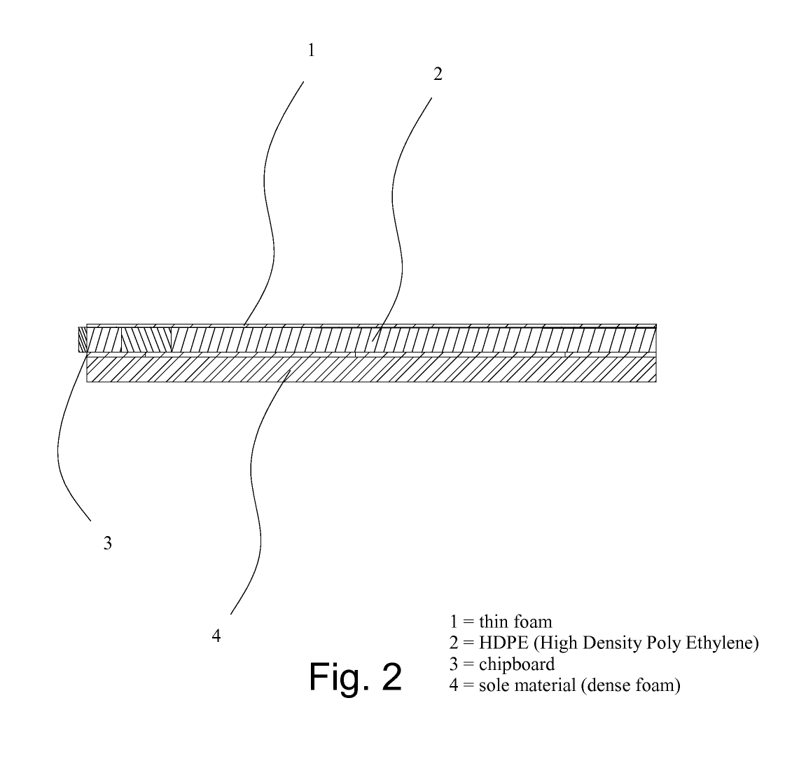

FIG. 3: Top view of side material, HDPE inset, foam covering, chipboard & sole material

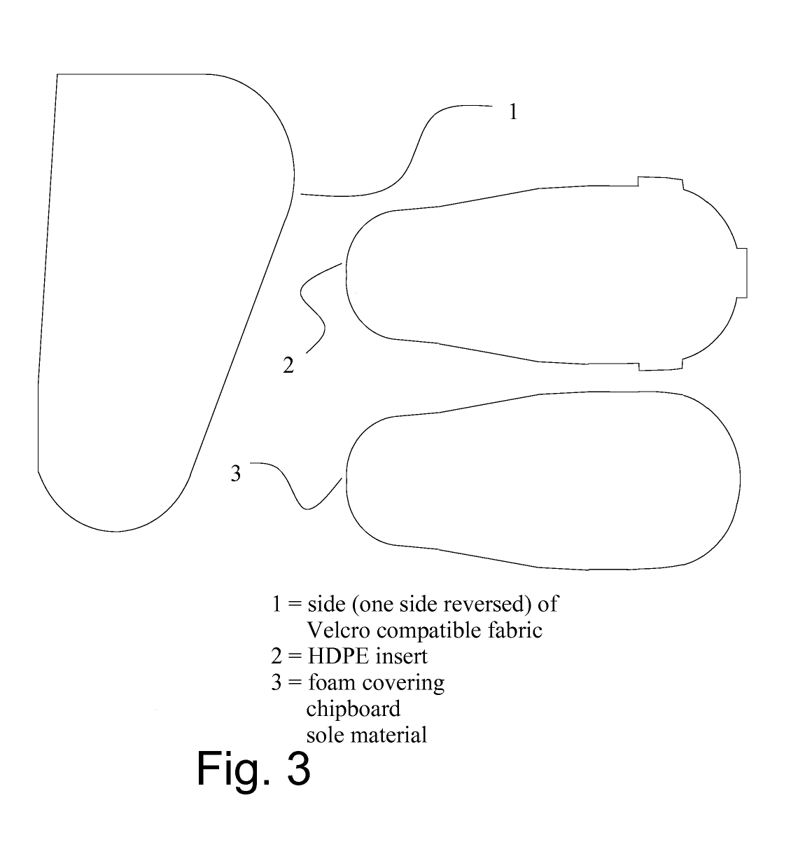

FIG. 4: Enlarged view of arch support

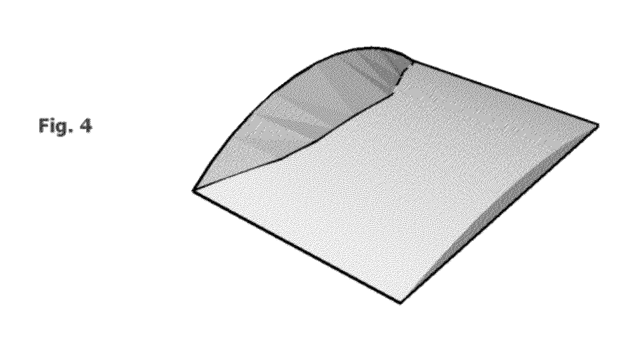

1. The Foot Protection & Alignment Device protects the toes and midfoot during the healing process. The device keeps the foot from flexing while being worn by way of a rigid insert. The arch support helps keep the foot in the proper position during healing. Keeping the foot in the proper position while healing eliminates, or minimizes, future problems. By wearing this device during the healing process of the foot, the patient minimizes risk of re-injury and promotes healing in the proper position.
